(19) United States
(12) Patent Application Publication
(10) Pub. No.: US 2021/0030789 A1
(43) Pub. Date: Feb. 4, 2021

(54) COMPOSITIONS AND METHODS FOR NEUROPROTECTION UTILIZING NANOPARTICULATE SILVER

(71) Applicant: Avalon Nanosilver Rx (HK) Limited, Central (HK)

(72) Inventors: Johnson Yiu-Nam LAU, Houston, TX (US); Michael Kai Tsun TO, Pokfulam (HK); Kenneth Man Chee CHEUNG, Mid Levels (HK)

(21) Appl. No.: 16/487,733

(22) PCT Filed: Feb. 21, 2018
(86) PCT No.: PCT/US2018/018923

§ 371 Date: Aug. 21, 2019

Related U.S. Application Data
(60) Provisional application No. 62/462,145, filed on Feb. 22, 2017.

(51) Int. Cl. A61K 33/38; A61K 9/14; A61K 38/50; A61P 25/28; A61K 47/38

(52) U.S. Cl. CPC A61K 33/38; A61K 9/14; A61K 47/38; A61P 25/28; A61K 38/50

(57) ABSTRACT

A preparation of silver nanoparticles has been found to be effective in improving functional and behavioral recovery from traumatic spinal cord injury. The silver nanoparticles are provided in a non-flowable gel vehicle, from which they are release at high efficiency, that is applied locally at the site of the spinal cord injury. Silver nanoparticle formulations described herein were found to modify the M1/M2 macrophage phenotype ratio and provides a synergistic effect in the combination with arginase to promote healing processes at the treated injury site, reducing postinjury inflammation.

This application claims the benefit of U.S. Provisional Application No. 62/462,145 filed on Feb. 22, 2017. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is prevention and/or treatment of neurological damage, particularly utilizing nanoparticulate metals.

### BACKGROUND

The background description includes information that may be useful in understanding the present invention. It is not an admission that any of the information provided herein is prior art or relevant to the presently claimed invention, or that any publication specifically or implicitly referenced is prior art.

Traumatic Spinal Cord Injury (TSCI) is a serious condition that can result in paralysis, a condition that dramatically impacts an affected individual's quality of life. As access to motor vehicles improves TSCI has become a global epidemic, with incidence rate of about 40 to 80 per million per year. Currently there are approximately 250,000 living survivors of TSCI in the United States. In China, there are about 78,000 people suffering from spinal cord injury and about 400 people in Hong Kong with severe chronic spinal cord injuries that are under care in rehabilitation centers. At a practical level, TSCI has a peak incidence in young adults that are otherwise healthy. As a result medical expenses accrued over the lifetime of one patient can amount of from about two to five million US dollars (depending on the severity of the injury).

TSCI is generally a result of traffic accidents, falls and sports injuries, and spinal cord damage during surgical procedures (for example, correction of spinal deformities). The sudden trauma to the vertebral column produces primary injury that compresses and damages the spinal cord, disconnecting the communication channel between the brain and the body, causing functional problems like sensory loss, neuropathic pain, lifetime paralysis and even death. In to the initial mechanical damage, the body responds to the injury with an inflammatory reaction that often results in a secondary injury. Such inflammatory reaction can result in ischemia, edema, excitotoxicity, hypoxia, disturbances of ion homeostasis and apoptosis. The process begins within minutes and evolves over several hours following the injury and manifests itself by neurologic deterioration over the first 8 to 12 hours in patients who initially present with an incomplete cord syndrome. Edema of the spinal cord will eventually be replaced by a central hemorrhagic necrosis that means irreversible neurological damage.

Current management of TSCI secondary injuries relies on administration of glucocorticoid (methylprednisolone), which is currently the only treatment option that has been found in clinical trials to improve outcome in TSCI patients (1-3). All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply. Despite glucocorticoid's anti-inflammatory effect many clinicians have raised concern about systemic effect of high dose glucocorticoid therapy, particularly in regards to immune responses to infection and in patients with moderate to severe traumatic brain/multisystem injury. It is also believed that glucocorticoid therapy can interfere with regenerative processes. Riluzole, as a neuroprotective drug, is currently under the investigation for reducing spinal cord damage (4-6). However, it is only useful if the drug is administered prior to injury to the spinal cord (4).

Silver nanoparticles (i.e. particles of silver metal having a mean diameter of less than 1 μm) have been found to have beneficial effects in various in vitro and in vivo studies directed to regenerative healing processes, specifically in skin, tendon and bone (11-13). Such studies have indicated that application of silver in the form of nanoparticles can result in improved collagen deposition in such tissues as they heal, and improved mechanical properties and thus functional outcomes of the repaired tissues.

Some studies have suggested that application of silver nanoparticles can reduce the expression of cytokines TGF-β1 and IL-6 (which have been associated with scar formation), while increasing expression of IL-10 and VEGF (which have been associated with matrix deposition and vascularization (14). Anti-inflammatory effects in certain tissues (such as peritoneum and Achilles tendon) have also been noted, with a reduction in neutrophil and macrophage infiltration, along with a reduction in TNF-α. At the same time production of GAG and various proteoglycans was enhanced. The mechanism behind these effects, however, are not clear.

Thus, there is still a need for safe and effective compositions and methods for treating the secondary effects of traumatic spinal cord injury.

### SUMMARY OF THE INVENTION

The inventive subject matter provides apparatus, systems and methods in which a preparation of silver nanoparticles (AgNPs), which can be provided as a hyaluronic acid containing gel, are applied to a site of central nervous system damage (for example, a spinal cord injury resulting from trauma due to accident and/or as a complication of spinal surgery) prior to, at the time of, or following trauma in order to reduce or prevent inflammation and aid in healing and recovery of function. Such AgNPs are found to modify the ratio between M1 and M2 phenotype macrophages, at least in part by selective killing of M1 phenotype cells.

One embodiment of the inventive concept is a method for treating neuronal tissue, in which a silver nanoparticle preparation is applied to a site in need of treatment, such as an injury prior to, at the time of, and/or following damage to the neuronal tissue (such as a site of an acute spinal cord injury, a site of a head injury, a site of injury to a nerve, and a site of stroke). Such treatment can provide neuroprotection prior to damage to the neuronal tissue, and improve recovery and/or healing if applied at the time of or following injury. The silver nanoparticles used have a mean diameter of less than about 1 μm (for example, about 20 nm to about 500 nm, about 500 nm to about 1,000 nm, or from about 5 nm to about 20 nm), and can be provided in a pharmaceutically acceptable carrier, for example a biopolymer such as hyalyuronic acid. The pharmaceutically acceptable carrier can be a liquid, a gel, a cream, an ointment, a paste, or an appliance. In some embodiments the AgNPs preparation is applied systemically. On other embodiments the AgNPs preparation is applied locally (e.g. at or near the site of injury). Such application can be performed within 48 to 96 hours from the time of the neurological injury. The AgNPs preparation can be applied in combination with a complementary therapy or therapeutic compound, for example a corticosteroid, a cytokine, or an antibody (e.g. a cell-specific or cytokine-specific antibody).

Another embodiment of the inventive concept is a method for modulating M1/M2 macrophage balance by applying a silver nanoparticle preparation to a site in need of protection from inflammation, such as a neuronal injury following damage to the neuronal tissue (such as a site of an acute spinal cord injury, a site of a head injury, a site of injury to a nerve, and a site of stroke). Such a method can provide neuroprotection if utilized prior to damage to the neuronal tissue. The silver nanoparticles used have a mean diameter of less than about 1 μm or from about 5 nm to about 20 nm, and can be provided in a pharmaceutically acceptable carrier, for example a biopolymer such as hyalyuronic acid. The pharmaceutically acceptable carrier can be a liquid, a gel, a cream, an ointment, a paste, or an appliance. In some embodiments the AgNPs preparation is applied systemically. On other embodiments the AgNPs preparation is applied locally (e.g. at or near the site of injury). Such application can be performed within 48 to 96 hours from the time of the neurological injury. The AgNPs preparation can be applied in combination with a complementary therapy or therapeutic compound, for example a corticosteroid, a cytokine, or an antibody (e.g. a cell-specific or cytokine-specific antibody).

Another embodiment of the inventive concept is a method for the application of the silver nanoparticle preparation in combination with arginase, which can provide a synergistic effect to the site in need of protection from inflammation, such as a prevention or treatment of neuronal injury resulting from damage to the neuronal tissue (e.g. a site of an acute spinal cord injury, a site of a head injury, a site of injury to a nerve, a site of stroke, and/or a site of surgical intervention). In some embodiments such treatment can provide neuroprotection if applied prior to damage to the neuronal tissue. The silver nanoparticles used have a mean diameter of less than about 1 μm or from about 5 nm to about 20 nm, and can be provided in a pharmaceutically acceptable carrier, for example a biopolymer such as hyalyuronic acid. The pharmaceutically acceptable carrier can be a liquid, a gel, a cream, an ointment, a paste, or an appliance. In some embodiments the AgNPs preparation is applied systemically. On other embodiments the AgNPs preparation is applied locally (e.g. at or near the site of injury). Such application can be performed within 48 to 96 hours from the time of the neurological injury. In some embodiments application can occur prior to the injury, for example prophylactic application at a site where spinal surgery is to be performed. The AgNPs preparation can be applied in combination with a complementary therapy or therapeutic compound, for example a corticosteroid, a cytokine, or an antibody (e.g. a cell-specific or cytokine-specific antibody).

Another embodiment of the inventive concept is a composition for use in treating an animal (which can include a human), which includes silver nanoparticles and a pharmaceutical carrier in the form of a non-flowable gel. The silver nanoparticles can have a mean diameter of less than about 1 μm, such as from about 5 nm to about 20 nm. The pharmaceutical carrier can include a biopolymer, such as a protein, a polysaccharide, a starch, and an aminoglycoside (e.g. hyaluronic acid). The pharmaceutical carrier can include a stabilizing agent that reduces or prevents aggregation of the silver nanoparticles, such as polyvinylpyrrolidone.

Another embodiment of the inventive concept is a kit for providing treatment of neuronal tissue, for example treatment of neuronal injury following damage to neuronal tissue. Such a kit includes silver nanoparticles having a mean diameter of less than about 1 μm (for example from about 5 nm to about 20 nm) and instructions for a treatment protocol that provides effective treatment. Such treatment can include providing neuroprotection prior to damage to the neuronal tissue (for example, as a result of surgical intervention at or near the spine), modulation of M1/M2 macrophage balance, and/or increasing arginase activity, and can be applied to a site of an acute spinal cord injury, a site of a head injury, a site of injury to a nerve, and/or a site of stroke. The kit can also include a pharmaceutically acceptable carrier, which can include a biopolymer. This pharmaceutically can be formulated as a liquid, a gel, a cream, an ointment, a paste, or an appliance. The kit can include one or more complementary therapeutics, such as arginase, a corticosteroid, a cytokine, and/or an antibody (such as a cell-specific antibody or a cytokine-specific antibody). Treatment protocols can provide systemic and/or local (i.e. at or near the treatment site) application of the silver nanoparticle within 48 to 96 hours of the time of the injury.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIGS. 1A and 1B: FIG. 1A shows typical results of immunofluorescence staining studies directed to the kinetics of neutrophil infiltration into a wound with and without treatment using silver nanoparticles (AgNPs). Green fluorescence represents Ly6G positive neutrophils; blue fluorescence represents DAPI-stained nuclei (200×). The degree of infiltration immediately post-injury and for up to 10 days afterwards is clearly reduced. FIG. 1B shows typical results of immunofluorescence staining studies directed to the kinetics of macrophage infiltration into a wound with and without treatment using silver nanoparticles (AgNPs). Green fluorescence represents F4/80 positive macrophages; blue fluorescence represents DAPI-stained nuclei (200×). The degree of infiltration immediately post-injury and for up to 10 days afterwards is clearly reduced.

FIGS. 2A and 2B: FIG. 2A2A and FIG. 2B show results of cell viability studies of original, unmodified phenotype RAW264.7 macrophages along withM1 and M2 phenotypes of RAW264.7 macrophages treated with different concentrations of 5 nm to 20 nm AgNPs on Day 2 and Day 3 of exposure, respectively. The selective killing effect of silver nanoparticles at concentrations of 20 μM and greater is evident.

FIGS. 3A and 3B: FIG. 3A schematically depicts competing pathways of L-arginine metabolism in M1 and M2 macrophages. In M1 macrophages NO is synthesized by iNOS from arginine, causing cytotoxicity and apoptosis. In M2 macrophages arginase produces polyamines and proline from L-arginine, which are beneficial to cell proliferation and collagen production. The arginase pathway is also involved in the urea cycle, which eliminates excess ammonia. FIG. 3B shows results of cell viability studies of RAW 264.7 expressing nonpolarized, M1, or M2 phenotypes on Day 2 of treatment with arginase (Hong Kong Polytechnic University). Arginase had no apparent affect on cell viability on nonpolarized, M1, M2 phenotypes in concentrations ranging from 0 ng/L to 1,000 ng/L.

FIG. 4: FIG. 4 schematically depicts a typical synthesis of an AgNPs-loaded hydrogel.

FIG. 5: FIG. 5 schematically depicts the mechanism utilized for production of controlled contusion spinal cord injuries (SCI) in the studies described herein.

FIGS. 6A to 6C: FIGS. 6A to 6C show the results of various studies characterizing the properties of AgNPs. FIG. 6A provides a photograph of a typical AgNPs suspension. FIG. 6B shows a typical UV-visible absorption spectrum of AgNPs. FIG. 6C6C shows the morphology and size distribution of AgNPs, based on TEM results.

FIG. 7: FIG. 7 shows the results of studies characterizing the in vitro release of AgNPs from hyaluronic acid/methyl cellulose (HAMC) hydrogels, in the absence of added solvents and/or releasing agents.

FIG. 8: FIG. 8 shows typical results of mobility studies of mice, using a forelimb locomotor scale (FLS) test to evaluate the effectiveness of AgNPs on behavioral outcome of mice following a C5-level spinal cord injury. Comparisons of FLS values between an AgNPs-treated group, control group, no hydrogel group, and sham group at 5 testing time points are shown.

FIG. 9: FIG. 9 shows typical results of mobility studies of mice, using a Ladder Rung Walking Performance test to evaluate the effectiveness of AgNPs on behavioral outcome of mice following a C5-level spinal cord injury with treatment of AgNPs. Shown are comparisons of missing ratio results between an AgNPs treated group, control group, no hydrogel group, and sham group at 4 testing time points.

FIGS. 10A and 10B: FIGS. 10A and 10B provide photomicrographs of histological studies of portions of injured spinal cord from AGNPs treated and control subjects. FIG. 10A shows typical results of H&E staining, with vacuolization indicated by a black arrow. FIG. 10B shows the degree of myelin loss in the injury site (black arrow), including posterior funiculus and dorsal horn. Presented as distribution and intensity of the color blue, relief of demyelination was shown as recovery in both groups.

FIGS. 11A and 11B: FIGS. 11A and 11B show typical results of immunofluorescent staining of portions of injured spinal cord from AGNPs treated and control subjects, using antibodies directed to inflammation markers. FIG. 11A shows typical results of immunofluorescence staining for the expression of TNF-alpha at the dorsal horn of spinal cord lesion sites in AgNPs group and blank hydrogel group at day 3 and day 9. Green fluorescence represents TNF-alpha expression. The mean intensity of fluorescence was quantified by software Image J. Values shown are means with one standard deviation. (Magnification: 10×. FIG. 11B shows typical results of immunofluorescence staining for the expression of iNOS at the dorsal horn of spinal cord lesion sites in AgNPs group and blank hydrogel group at day 3 and day 9. Green fluorescence represents iNOS expression and the mean intensity of fluorescence were quantified by software Image J. (Magnification: 10×)

### DETAILED DESCRIPTION

The following description includes information that may be useful in understanding the present invention. It is not an admission that any of the information provided herein is prior art or relevant to the presently claimed invention, or that any publication specifically or implicitly referenced is prior art.

The inventive subject matter provides apparatus, systems and methods in which a silver metal is provided as nanoparticles having a mean diameter of less than about 1 μm, less than about 500 nm, or between about 5 nm and about 999 nm, where such nanoparticles are applied systemically or locally at the site following a traumatic neurological injury (such as a traumatic spinal cord injury) in order to treat or prevent the effects of the neurological injury. Inventors believe that neuroprotective effects are provided by an alteration of the response of M1 and M2 macrophages at or near the site of the injury that, in turn, reduces inflammatory responses at or near the site of injury. Specifically, Inventors believe that M1 macrophages are preferentially targeted by nanoparticulate silver, and that such targeting results in inactivation or death of such M1 macrophages. Surprisingly, Inventors have found that such nanoparticles can effectively improve recovery in animal models of traumatic spinal cord injury when applied to the site of injury as a suspension in a hyaluronic acid-based gel formulation, from which they are released efficiently without the use of co-solvents or other releasing agents.

Silver nanoparticles of the inventive concept are particles of metallic silver (such as those produced by reduction of soluble silver salts). These are typically essentially monodisperse and have a mean diameter of less than 1 μm. Suitable mean diameters for such nanoparticles are between about 5 nm and about 999 nm, for example about 5 nm, 10 nm, 15 nm, 20 nm, 25 nm, 30 nm, 40 nm 50 nm, 75 nm, 100 nm, 200 nm, 300 nm, 400 nm, 500 nm, 600 nm, 700 nm, 800 nm, and 900 nm. In a typical formulation, the majority of silver nanoparticles have a diameter ranging from about 5 nm to about 20 nm.

In compositions of the inventive concept silver nanoparticles can be in the form of a suspension or dispersion in a liquid, gel, paste, cream, or other fluid or semi-fluid carrier. In some embodiments silver nanoparticles are provided in a solid carrier that dissolves or disperses over time. Such carriers can be made of any pharmaceutically acceptable material, such as a biopolymer (e.g. a protein, polysaccharide, starch, glycosaminoglycan, etc.). In a preferred embodiment the suspension or dispersion include hyaluronic acid, which can be used in combination with one or more other biopolymers.

Hyaluronic acid is a component of the extracellular matrix, and has been utilized clinically in replacement or supplementation of synovial fluid in arthritic joints in an attempt to improve function. Unfortunately no clear clinical benefit has been demonstrated for such treatments, which in some instances have generated significant negative side effects. Hyaluronic acid has also been utilized in dermal fillers that are injected subdermally for cosmetic purposes. Unfortunately such uses have been associated with inflammatory reactions and foreign body-type granulomatous reactions. It should be appreciated that the presence of hyaluronic acid is also normally associated with increased inflammation. The effectiveness of silver nanoparticles in reducing inflammation and promoting recovery from central nervous system injury (as shown below) is, therefore, counterintuitive and unexpected in light of the role of inflammation in post-trauma damage in such injuries.

In methods of the inventive concept, preparations that contain silver nanoparticles can be applied systemically or locally. Suitable methods for systemic application include injection and oral ingestion. Local application can be provided by direct application of a fluid suspension, injection (for example, of a thickened or viscous preparation), application of a semi-solid (e.g. gel, paste, and/or cream), and/or application or positioning of an implant or appliance that includes silver nanoparticles at or near a site in need of treatment.

One should appreciate that the disclosed techniques provide many advantageous technical effects including safely and effectively reducing damaging inflammation that typically follows an acute injury, particularly to the central nervous system, while supporting subsequent healing and/or regeneration.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

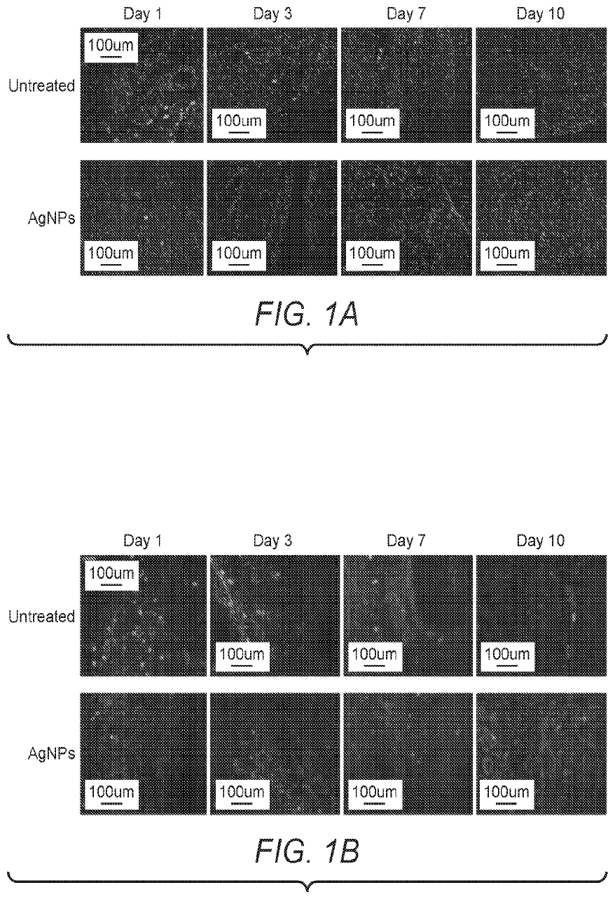

One embodiment of the inventive concept is the use of nanoparticulate silver to modulate the balance between M1 and M2 macrophages. M1 macrophages are associated with inflammatory processes (which are responsible for significant damage following spinal cord injury), whereas M2 macrophages are associated with cell recruitment and regeneration. In a preferred embodiment this modulation in the balance between M1 and M2 macrophages can be used to reduce and/or prevent inflammation, in particular inflammation associated with or leading to neurological trauma. As shown in FIGS. 1A and 1B, treatment of injured tissue (in this instance tendon) with silver nanoparticles results in a reduction in infiltration by neutrophils and/or macrophages, which are associated with inflammatory processes.

FIG. 1A shows the results of immunofluorescent staining for Ly6G positive neutrophils (nuclei are counterstained with DAPI) at 200× magnification, and shows the kinetics of such neutrophil infiltration into a wound with and without treatment using silver nanoparticles (AgNPs). Similar studies are shown in FIG. 1B, in which immunofluorescent staining was performed for F4/80 positive macrophages. The degree of infiltration immediately post-injury and for up to 10 days afterwards is reduced. This was also shown in spinal cord injury experiments, as shown below.

Inventors believe that silver nanoparticles can result in selective cell death of macrophages, which can lead to a modulation (such as a reduction) in the M1/M2 ratio found in acute inflammatory responses (such as those following neurological injury). In addition, Inventors have surprisingly found that application of silver nanoparticles can modulate the M1/M2 ratio in acute inflammation, such as that following neurological trauma (e.g. spinal cord injury, stroke, etc.). Without wishing to be bound by theory, Inventors believe that AgNPs can be selectively cytotoxic for certain cell types. Inventors have identified differential effects of AgNPs on primary chondrocytes and rat chondrosarcoma (RCS) cells, where the cytotoxicity of AgNPs was found to be dose-dependent. Notably, primary RCS cells demonstrated decreased viability at concentrations higher than 20 μM. Primary chondrocytes were found to tolerate higher AgNPs concentration. Beyond cytotoxicity, proteoglycan expression in RCS cells cultured at 20 μM AgNPs showed that the expression of fibromodulin is up-regulated and that there are observable morphological changes at the nucleus near the Golgi apparatus that are indicative of an increased level of cell secretory activities.

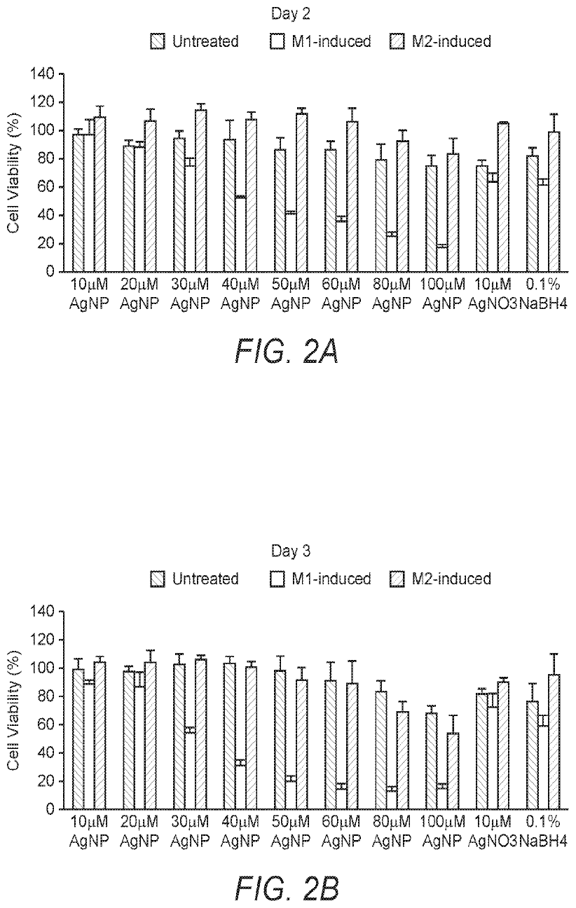

To further characterize such selective cytotoxicity, Inventors studied the effects of AgNP exposure on RAW264.7 cells. These cells provide an experimental model often used for studying macrophage polarization, as the in vitro polarization of this cell line is well established. RAW264.7 cells treated with 100 ng/mL LPS plus 2.5 ng/mL IFNγ develop an M1 phenotype, whereas treatment with 10 ng/ml IL-4 results in an M2 phenotype. MTT staining (indicative of cell metabolic activity) showed that cells having an M1 phenotype showed significantly reduced viability compared to those with the M2 phenotype when exposed to 20 μM AgNPs having a diameter ranging from about 5 nm to about 20 nm. FIGS. 2A and 2B show results of cell viability studies of M1 and M2 phenotype RAW264.7 macrophages treated with different concentrations of 5 nm to 20 nm AgNPs on day 2 (FIG. 2A) and day 3 (FIG. 2B) of exposure, showing a selective killing effect of silver nanoparticles at concentrations of about 20 μM or higher.

Modulation (e.g. reduction) in the M1/M2 ratio of macrophages present in the site of traumatic injury by the application of silver nanoparticles can be useful in treatment and/or prevention of negative outcomes of such traumatic injuries. Specifically, reduction in the M1/M2 ratio following traumatic injury by application of silver nanoparticles can selectively reduce damaging inflammatory effects while not resulting in general cytotoxicity and/or other negative effects of current treatment protocols. In preferred embodiments, the traumatic injuries are related to central nervous system trauma (e.g. spinal cord injuries, head injuries, strokes, etc.) and the silver nanoparticles provide a neuroprotective effect.

The pathophysiology of traumatic spinal cord injury (TSCI) includes both primary and secondary injury mechanisms that coordinate to result in the development of a necrotic core of tissue that forms a cavity within the spinal cord during the first week post-injury (27-29). Secondary injury expands the damage to levels rostral and caudal to the actual impact site in the form of ischemia, edema, increased excitatory amino acids and oxidative damage to the tissue from reactive oxygen species (28, 30, 31). Improved emergency care of people with spinal cord injuries and aggressive treatment and rehabilitation can minimize damage to the nervous system and even restore limited abilities.

The mechanisms of the TSCI primary injury are characterized by (i) impact plus persistent compression; (ii) impact alone with transient compression; (iii) distraction; and (iv) laceration/transection (32). The initial mechanical insult tends to damage primarily the central gray matter, with relative sparing of the peripheral white matter. The injured spinal cord suffers early hemorrhage and later develops disrupted blood flow. Such disruption results in local infarction caused by hypoxia and ischemia. This is particularly damaging to the grey matter due to its high metabolic requirement (32). Neurons that pass through the injury site are physically disrupted and exhibit diminished myelin thickness (34). Nerve transmission can be disrupted further by edema and micro-hemorrhages near the site of injury (32, 35-37). Gray matter suffers irreversible damage within the first hour of injury while white matters within 72 hours (38), providing a limited window of opportunity for effective treatment Inflammatory response induced immunologic damage as a result of recruitment and activation of immune cells has been widely accepted as a substantial contributor to the secondary damage of spinal cord (28, 39-41).

The acute inflammatory response to injury involves recruitment of neutrophils and macrophages to the site of injury. Microglia of the central nervous system also play in important role in initiation of the inflammatory response. Such a response almost immediate follows the occurrence of a TSCI with production of pro-inflammatory cytokines and chemo-attractants by cells such as endothelial cells within the damaged tissues (28, 42, 43). This in turn enhances endothelial cell expression of adhesion molecules (ICAM-1, VCAM-1) which allows neutrophils to bind through counter-receptors (LFA-1, VLA-4) and to migrate into the tissue within a few hours of injury (44, 45). An influx of macrophages follows. Neutrophils and macrophages induce an oxidative burst resulting in the production of reactive oxygen species during phagocytosis of debris (28, 35). These reactive oxygen species can cause substantial secondary damaged by mediating lipid peroxidation and protein nitration, and by activating redox-sensitive signaling cascades and consumption of nitric oxide (39, 46). Surrounding, relatively healthy, tissue can be damaged as a result. Damage due to such inflammatory responses can also occur following cerebral contusion (47, 48), stroke involving ischemia and reperfusion (49, 50) and severance of the optic nerve (51, 52).

Macrophages and microglia are considered to be integral component of neural regeneration. Macrophages in the injured spinal cord are derived from blood-borne monocytes and resident microglia (60). In a rat model, blood-borne monocytes infiltrate the lesion within 2 days after the initial injury, achieve highest density at 5-7 days, and persist for weeks to months (28). Microglia can be activated within minutes to hours after the injury and are transformed into macrophages (61).

Macrophages have been shown to exhibit great plasticity and can alter their phenotypes and functions according to changes in stimuli provided by their microenvironment (40). Several macrophage subsets with distinct functions have been reported, including M1, M2 , regulatory macrophages, tumor associated macrophages, and myeloid-derived suppressor cells and others (64). It has been observed that the majority of macrophages accumulated following TSCI are M1 (65).

M1 macrophages are known to produce deleterious effects following TSCI as they produce high level of IL-1beta, IL-6, IL-12, IL-23, CCLS, TNF-alpha, IFN-sigma and iNOS (66, 67). M1 macrophages also express high levels of leukotriene B4 and prostaglandins, mediators of inflammation and secondary injury, relative to M2 macrophages (68). M2 macrophages, on the other hand, have been shown to participate in healing and repair processes (72). Inventors believe that reducing M1 macrophage proliferation and providing inhibition and/or suppression of M1 expression within the injured zone of the spinal cord can control and/or resolve damaging inflammation after TCSI, and that such can be provided by application of AgNPs.

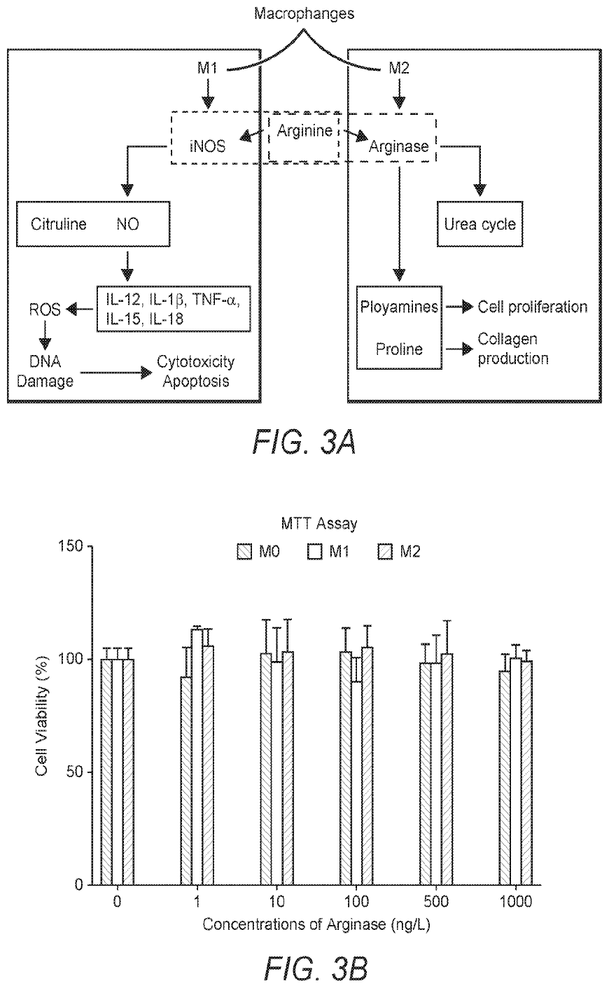

The Inventors note that expression of arginase has been associated with recovery from central nervous system injuries. During an inflammatory response arginase is predominantly produced by M2 macrophages activated through alternate pathways, while classically activated M1 macrophages express inducible nitric oxide synthase (iNOS). Both arginase and iNOS are involved in L-arginine metabolism. Arginase hydrolyzes L-arginine to produce ornithine (a precursor to proline and polyamines), stimulating cell proliferation, and urea (which aids in removing excess ammonia). In some conditions iNOS can compete for available L-arginine to produce NO, a cytotoxic mediator that can oxidize DNA, proteins, and/or lipids and thereby produce deleterious effects. Competition between arginase and iNOS is shown schematically in FIG. 3A. As shown in FIG. 3A iNOS produced by M1 macrophages reacts with L-arginine to produce NO, resulting in cytotoxicity and apoptosis. M2 macrophages produce arginase, which reacts with L-arginine to produce polyamines and proline. These in turn support cell proliferation and collagen production. In addition, Inventors believe that treatment with AgNPs in combination with arginase at the applied site of tissue injury can provide a synergistic effect that further enhances the healing effect. IWithout wishing to be bound by theory, the Inventors believe that provision of additional arginase at a treated site can speed up the break down of arginine into polyamines and proline, which in turn can further promote cell proliferation and collagen production during the recovery process.

FIG. 3B shows the correlation between the concentration of applied arginase and the viability of RAW 264.7 cells expressing nonpolarized, M1, and M2 phenotypes. As shown arginase applied over a wide range of concentrations has no impact on cell viability (as determined by MTT assay). Accordingly, it is expected that the use of AgNPs in combination with arginase should not adversely affect cell viability at treated sites. Arginase for these studies was provided by Hong Kong Polytechnic University, in accordance with U.S. Pat. No. 8,507,245 (which is incorporated herein by reference).

Surprisingly, Inventors have found that silver nanoparticles can demonstrate selective killing and/or inhibition of M1 macrophages while preserving M2 macrophages, and that such a modulation in the balance between these macrophage activities can be very useful in treating TSCI. It should be appreciated that conventional approaches do not provide a single agent that can reduce the deleterious effect of M1 macrophage activity in spinal cord injury while promoting M2 macrophage activity (which can provide a positive effect in spinal cord regeneration).

In one embodiment of the inventive concept, silver nanoparticles are utilized in the treatment of traumatic spinal cord injuries. In a preferred embodiment such treatment results in an improvement of at least motor recovery relative to a sham treatment lacking silver nanoparticles. Inventors have found, surprisingly, that silver nanoparticles can enhance motor recovery in rats after TSCI. Without wishing to be bound by theory, Inventors believe that silver nanoparticles can reduce or alleviate the acute inflammation that normally follows acute spinal cord injury, and protect the spinal cord from secondary injury by modulating the activities of immune cells (macrophages, microglia, neutrophils) while also activating and signaling them to commence regenerative processes that result in accelerated healing. The utility of silver nanoparticle preparations in treatment of animal models of impact-induced TSCI rat-models and the resulting enhancement of motor recovery are provided in the examples below.

In some embodiments of the inventive concept silver nanoparticles are used in combination with complementary therapeutic approaches to provide neuroprotection and/or treat neurological injury. Suitable complementary therapeutic approaches include treatment with arginase and/or anti-inflammatory compounds, such as corticosteroids and/or NSAIDs. Other complementary therapeutic approaches include treatment with specific antibodies (e.g. monoclonal antibodies) directed to components of the immune system and/or inflammatory process. Examples include antibodies directed to M1 macrophages, interleukin 1, γ-interferon, and/or TNFα. Still other complementary therapeutic approaches include treatment with cytokines that favor an increased M2 component in the M1/M2 balance. Such cytokines include interleukin 4, interleukin 10, and interleukin 13.

### EXAMPLES

Preparation of silver nanoparticle (AgNP)-loaded hyaluronic acid/methylcellulose (HAMC) hydrogel: Silver nanoparticles (AgNPs) in suspension were synthesized using a chemical reduction method. A 500 ml solution containing 0.7 mM sodium citrate dihydrate (Sigma-Aldrich) and 0.1 mM silver nitrate (AgNO₃; Sigma-Aldrich) was bubbled and vigorously stirred under nitrogen for 30 minutes at room temperature. To this was added 0.5 ml 10 mg/ml sodium borohydride (NaBH₄; Sigma-Aldrich) solution, followed by stirring for 4 hours in the dark (to completion). After overnight ageing, the solution was concentrated from 500 ml to 50 ml by rotary evaporation and 50 μl of 100 mg/ml polyvinylpyrrolidone (PVP; Mr=10,000 Da; Sigma-Aldrich) added as a stabilizer to prevent AgNPs from aggregating during storage. The final concentration of AgNPs suspension obtained was approximately 1 mM (in terms of Ag mass per volume).

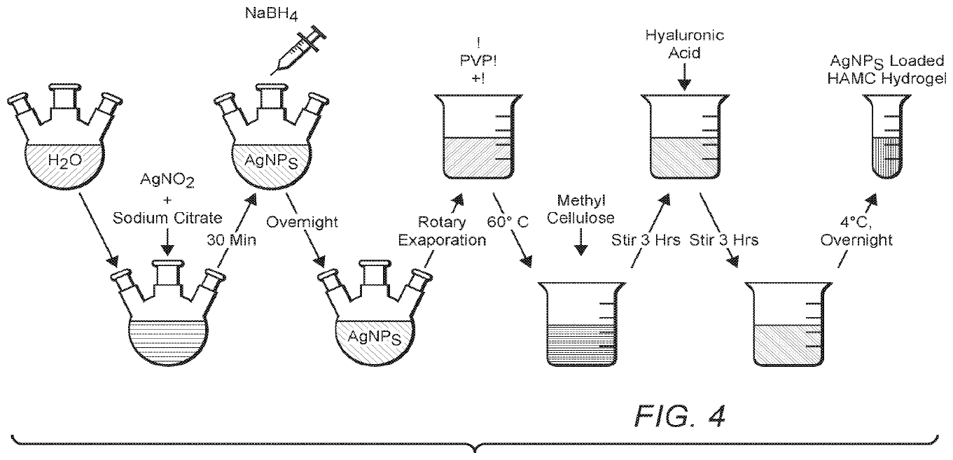

A hyaluronic acid/carboxymethylcellulose (HAMC) hydrogel was prepared in sterile artificial cerebrospinal fluid (aCSF; pH of 7.4; Composition: 148 mM NaCl, 3 mM KCl, 0.8 mM MgCl₂, 1.4 mM CaCl₂, 1.5 mM Na₂HPO₄, and 0.2 mM NaH₂PO₄) using 2 wt % sodium hyaluronate (HA; MW=2,600 Da; Ryon) and 5 wt % methylcellulose (MC; MW=300 Da; Sigma-Aldrich). Sterile MC and HA were sequentially and mechanically dispersed in the aCSF at 60° C. and allowed to dissolve at 4° C. overnight. AgNPs-loaded HAMC hydrogels were similarly prepared by sequentially and mechanically distributing 5 wt % MC and 2 wt % HA into a sterile AgNPs solution and allowing the suspension to dissolve at 4° C. overnight. A typical synthesis is shown schematically in FIG. 4.

Characterization of AgNPs: The size and morphology of the synthesized AgNPs were analyzed by transmission electron microscope (TEM; Philips Technai 12) at 120 KVs. Absorption across the UV-visible spectra was also recorded using a spectrophotometer by Full-wave UV spectra scanning (NanoDrop 2000; Thermo Scientific).

Release of AgNPs from HAMC hydrogels: Cumulative release profiles of silver nanoparticles from HAMC hydrogel preparations were measured in vitro over a sequential set of time periods. Specifically, portions of AgNPs-loaded hydrogel weighing about 1 g were stored in 10 ml aCSF (releasing medium), and oscillated at a frequency of 60 rpm on a rotary shaker at 37° C. At various time points 0.5 ml of the releasing medium was extracted and stored at 4° C. for further characterization. At such time points 0.5 ml of fresh aCSF was added to the releasing medium. The amount of silver nanoparticles released was measured by optical density (O.D.) at 400 nm, using a spectrophotometer (NanoDrop 2000; Thermo Scientific).

Animal Studies: Forty-nine eight-week-old Sprague-Dawley (SD) rats, weighing around 250 grams, were randomly divided into 4 groups, with 20 rats in the AgNPs group, 18 rats in the blank hydrogel group, 5 rats in the no hydrogel group, and 6 rats in the sham group. Rats in the control group were injected with blank, unloaded HAMC hydrogel, while rats in AgNPs test group were injected with AgNPs-loaded HAMC hydrogel after contusion surgery. No hydrogel group rats were injected only with saline after contusion surgery and the spinal cord of animals in the sham group were surgically exposed but not contused. In each group, the rats were euthanized for H&E stain and Luxol fast blue stain at 3 different time points after introduction of injury through surgical operation (at the end of 7, 9 and 14 days) and for immunohistochemical analysis at 2 different time points (at the end of 1 and 9 days). The animals were obtained from the Laboratory Animal Unit, the University of Hong Kong and provided diet and water ad libitum in a room with alternating periods of 12 hours light and 12 hours dark.

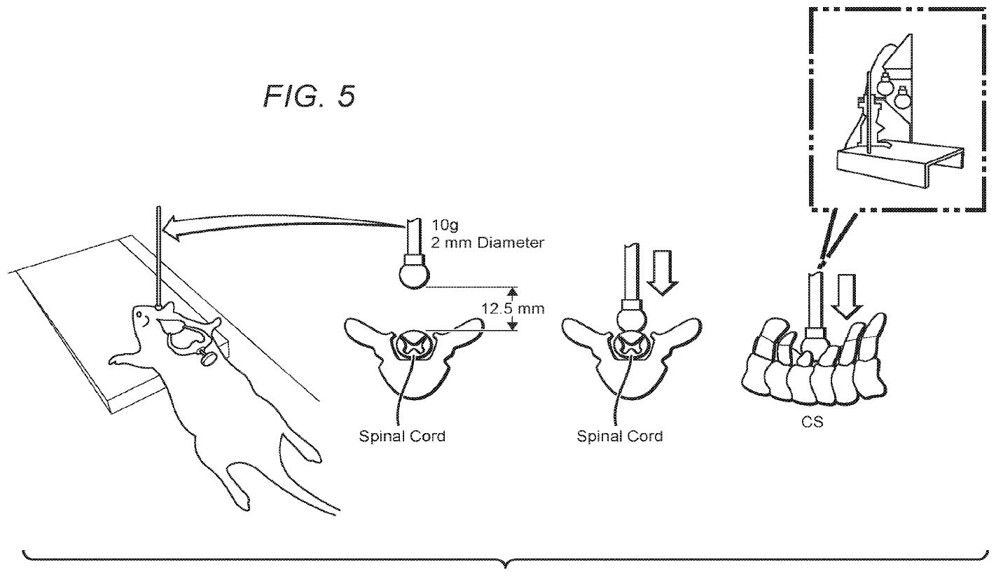

Contusive spinal cord injury: The rats were operated on under general anesthesia with ketamine/xylazine mixture (K:X=2:1) intraperitoneally. A skin incision was made along the sagittal plane to expose the muscle covering the lamina along C3-C7. Subsequently, laminectomy was performed with microsurgery tools at vertebral level C5 to expose the spinal cord. As illustrated in FIG. 5, a NYU-MASCIS Contusion spinal cord injury (SCI) was made using a customized weight-drop injury device modified from NYU impactor device (85, 86). Specifically, a 10-gram sterile blunt metal rod of 2 mm diameter was carefully lowered until it made contact with the dura. To imitate incomplete acute SCI through impact, the rod was freely dropped on the left part of spinal cord at the C5 level from a height of 12.5 centimeters in order to create observable clinic signs on impaired locomotion of left forepaws. The incision site was subsequently sutured layer by layer. Inventors believe that this model provides an accurate simulation of traumatic spinal cord injury due to both accidents and trauma induced by spinal surgery.

Treatment with AgNPs: Before the incision site was sutured layer by layer, for rats in AgNPs group, 0.1 ml 1 mM sterile AgNPs solution was directly instilled on the injury site, which was followed by injection of 0.15 ml AgNPs-loaded hydrogel onto the dura of vertebral level C5. For rats in the control group, 0.1 ml warm injectable saline as well as 0.15 ml blank HAMC hydrogel were sequentially placed on the injury site. Overall, the total dosage of AgNPs delivered into the injury site was controlled at about 45 μg/kg. During the first three days of recovery analgesic medication (buprenorphine, 0.01 to 0.02 mg/kg) was administrated through intramuscular injection two times a day. Animals were carefully monitored for signs of pain, inflammation, and any other post-operative complication. Inventors note that similar effects would be expected from performing such a procedure in the presence of the AgNP formulation, which would essentially provide immediate application of the formulation to the site of the injury and conclude that, logically, such application would provide a protective effect against subsequent inflammation and injury.

The assessment of functional recovery was conducted using both a Forelimb Locomotor Scale (FLS) test (87) and a Ladder Rung Walking Test (88). Time points for observation were at the 3rd day, 5th day, 7th day, 9th day and 14th day post operation, respectively.

The FLS was designed to give a quick observational score that describes a forelimb's functional capability during locomotion (87). The categories of scoring system are based on behavioral changes observed after unilateral cervical injury, with a range from 0 (complete paralysis) to 17 (healthy condition). Rats were placed in an enclosure (5 cm x 1 m), allowing the animal to move freely. Left forepaw behaviors were recorded by digital video for further analysis by two blinded observers. Each rat performed the test trials 4 times at each time point after 1 or 2 warm-up practices.

The Ladder Rung Walking Test was employed to evaluate the changes in the motor function of affected paws during recovery, and is regarded as a being more objective, quantitative, and sensitive to subtle changes in performance (88). Briefly, for all time points except the 3rd day, the animals were offered a task to reach their home cage by crossing a horizontally-placed ladder with metal rungs (3 mm diameter) randomly. The rats' performances on the ladder were video-recorded and analyzed frame by frame by two blinded observers. Steps with their left forepaws were scored on the basis of a 7-grade scale and further dichotomized as error (0-2) or correct steps (3-7). The “missing ratio” was defined as the number of any kind of foot slips or total misses among the 10 steps in the middle of a trial. The mean numbers of error steps for 5 trials were calculated for comparison.

Histological staining and Immunohistochemistry: The rats were sacrificed at the indicated time points using an overdose of sodium pentobarbital (100 mg/kg) and perfused intra-cardially with 250 ml saline, following by perfusion of 200 ml 4% paraformaldehyde in phosphate buffer (pH 7.4). Spinal cord segments at the C5 level were obtained and fixed in 4% paraformaldehyde overnight. Samples used for histological staining were subjected to dehydration and decalcification, and the processed pieces of tissue were embedded in paraffin and sectioned. 6-μm paraffin embedded transverse sections of injured spinal cord tissue were prepared for histological analysis. Samples used for immunohistochemical (IHC) staining were sequentially dehydrated in 30% sucrose solution at 4° C. overnight and embedded in optimal cutting temperature compound (OCT compound) prior to frozen sectioning.

To observe the impact of AgNPs on the inflammatory response at the lesion site, especially the manipulation of two phenotypes of macrophage (M1 and M2), Arg-1 antibody (1:50, a biomarker for M2 phenotype, GeneTex) or TNF-alpha antibody (1:50, a biomarker for M1 phenotype, Abcam), and iNOS antibody (1:50, a biomarker for M1 phenotype, Abcam) were used as primary antibodies in immunohistochemistry studies, along with appropriate AlexaFluor conjugated secondary antibodies (1:200; Life Technologies Corp.) and Hoechst 33342 (Sigma-Aldrich) for better visualization.

Myelin integrity: Myelin integrity was qualitatively and quantitatively determined in paraffin-embedded sections that were stained with Luxol fast blue for myelin and with cresyl violet for Nissl substance of the neurons and cell nuclei. For microscopic analysis, a Nikon fluorescent microscope (Nikon E800) was used. To determine histological morphology and pathological changes at the lesion site demarcation of the damaged site was performed using H&E staining.

Statistical Analysis: Statistical analysis was conducted to characterize differences between the AgNPs group and control groups and among various time points in recovery. To analyze the influence of the two main factors comprised of group (AgNPs and control groups) and time points (3rd, 5th, 7th, 9th and 14th day) on FLS scores, two-factor ANOVA was utilized as a statistical model. In the following, a t test was applied to test the variance of FLS scores between two groups at the same time point while a Bonferroni post hoc test for multiple comparisons was used to test the difference between various time points.

Similarly, the influence of various factors on experimental groups (AgNPs and control groups) and observation time points (5th, 7th, 9th and 14th day) on missing ratios were characterized by two-factor ANOVA as a statistical model, which was following by t test to test the difference of missing ratios between two groups at the same time point and Bonferroni post hoc to test the difference among various time points.

### Results

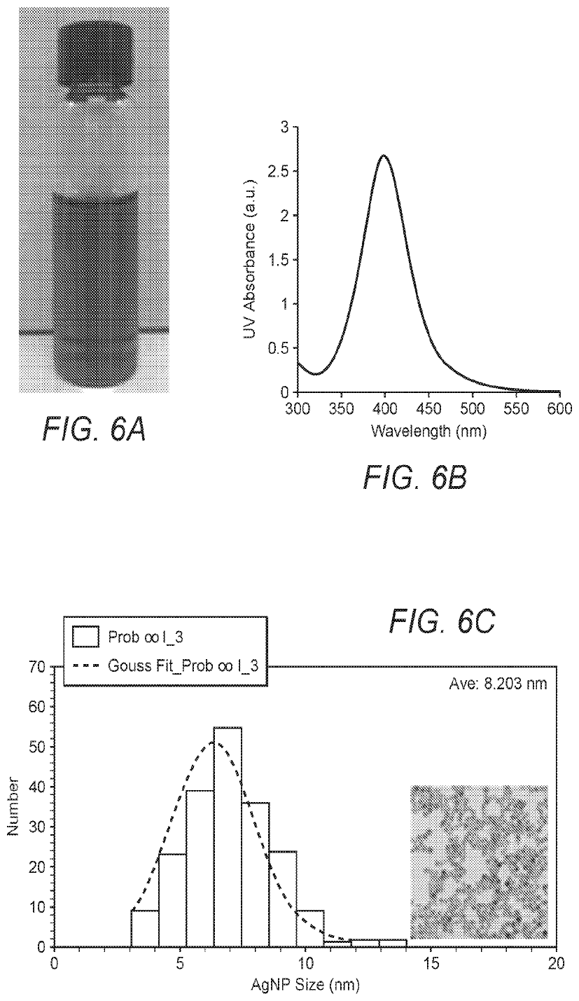

Characterization of AgNPs: The TEM image (see FIG. 6A) shows that the AgNPs are spherical and monodisperse in water. As shown in FIG. 6C, the diameter of the majority of the nanoparticles was distributed from about 5 nm to about 12 nm, with a median diameter of 8.203 nm. UV-visible absorption spectrum of the synthesized AgNPs solution (see FIG. 6B) exhibited an absorbance peak at 400 nm, which confirmed the presence of nanoparticles in the solution.

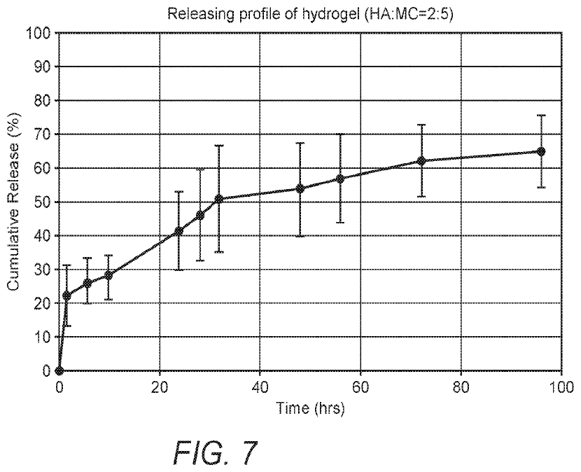

Release profile of AgNPs from HAMC hydrogels: The results of in vitro studies of the release of AgNPs from HAMC (HA:MC=2:5) hydrogel over 5 days are been shown in FIG. 7. About 108 μg/gram AgNPs were loaded in the HAMC hydrogel, and the drug (AgNP) release from delivery system was greater than 65% of the initial nanoparticle load after 5 days. This suggests that a large percentage (e.g. greater than 10%, 20%, 30%, 40%, 50, 60%, or by weight) of the AgNPs present in the hydrogel would be locally delivered at the site of injection.

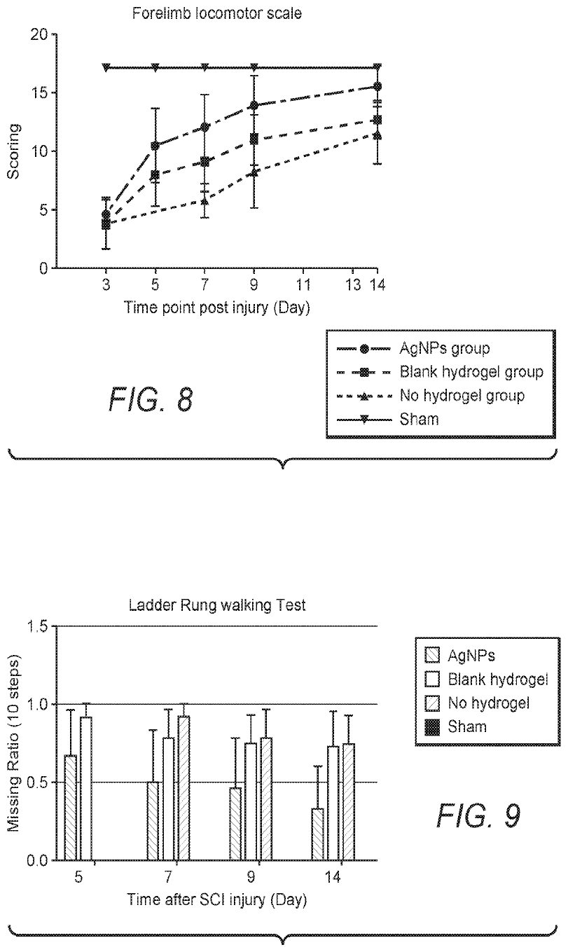

Assessment of motor function: Results for FLS testing of recovery are shown in FIG. 8. Both of the factors of treatment group and time point were found to have a significant influence on the FLS score. For the AgNPs group the differences between day 3 and day 5 were remarkably significant (p<0.01), as same as control group. At day 7, the AgNPs group were found to have a significantly greater FLS than the blank hydrogel (p=0.033) and the no hydrogel groups (p<0.01). At day 9, the AgNPs group showed remarkably greater FLS values than the blank hydrogel group (p=0.048) and the no hydrogel (p<0.01). At day 14, the AgNPs group showed significantly larger FLS values than the blank hydrogel group (p=0.033) and the no hydrogel (p=0.015).

In general, the AgNPs group demonstrated significantly larger values for the FLS score than the control group. There were no significant differences between the blank hydrogel and the no hydrogel group at each time point. The sham group (with no spinal cord injury) showed significantly larger FLS than the other three groups at all time points. According to t tests, the

FLS scores between two groups were remarkably different at the 7th day (p=0.033), 9th day (p<0.01), 14th day (p=0.015) post operation.

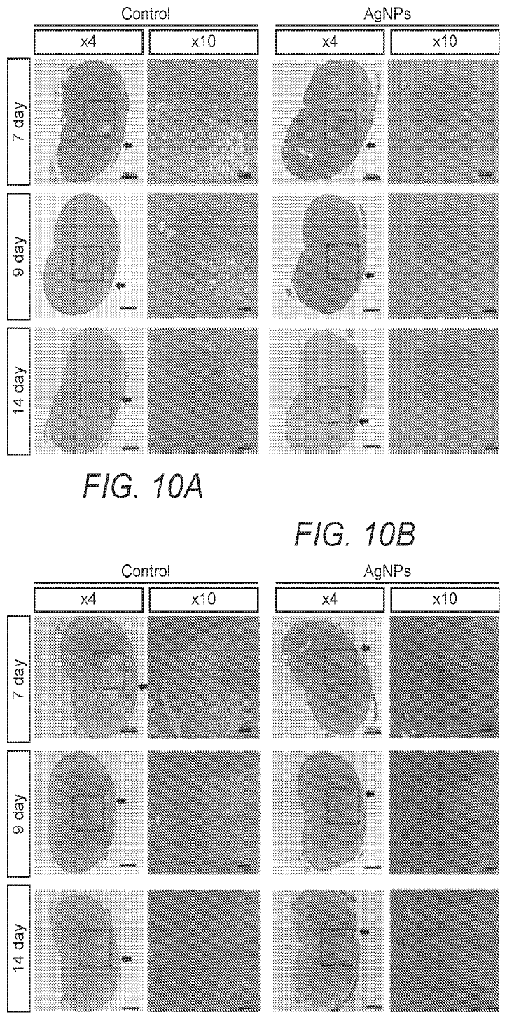

Results for Ladder rung walking testing of recovery are shown in FIG. 9. Both of the factors of treatment group and time point showed notable influences on the missing ratio observed during the Ladder rung walking test. There was a decrease in the missing ratio as recovery progressed for each of the two groups.

In comparing the missing ratios observed for the AgNPs and the blank hydrogel groups at same time point, the values for the blank hydrogel group are significantly larger than those of the AgNP group. At day 7, the AgNPs group showed remarkably smaller missing ratios than the blank hydrogel group (p=0.019) and the no hydrogel group (p=0.018). At day 9, the AgNPs group showed significantly smaller missing ratios than the blank hydrogel group (p=0.033) and the no hydrogel group (p=0.025). At day 14, the AgNPs group showed significantly smaller missing ratios than the blank hydrogel group (p=0.016) and the no hydrogel group (p<0.01). There were no significant differences between blank hydrogel group and the no hydrogel group at any time point. The sham group had a significantly smaller missing ratio than other three groups at all time points.

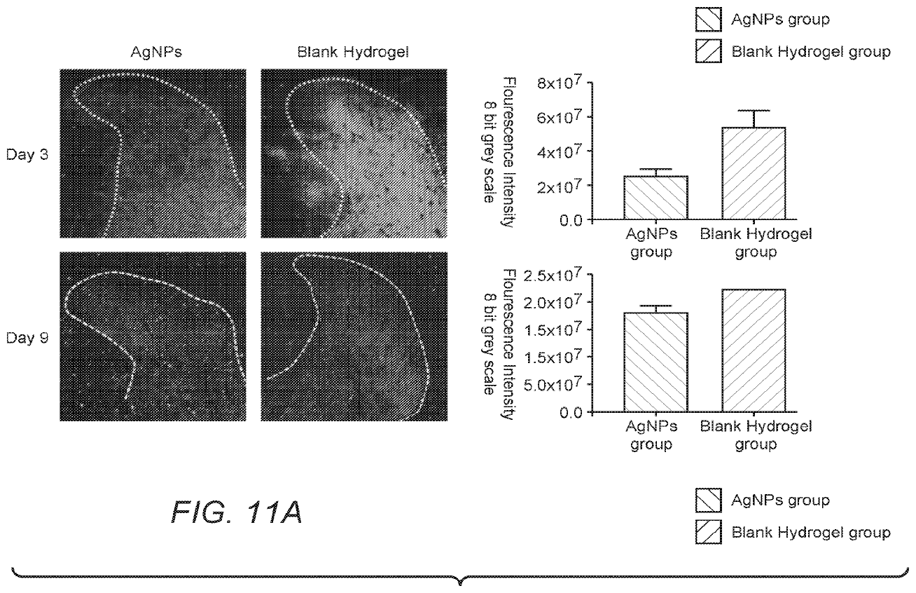

Histology assessment: Typical results of histology studies are shown in FIGS. 10A and 10B. FIG. 10A shows typical results of H&E staining, with vacuolization indicated by a black arrow. FIG. 10B illustrates the degree of myelin loss in the injury site (black arrow), including posterior funiculus and dorsal horn. Presented as distribution and intensity of the color blue, relief of demyelination was shown as recovery in both two groups, respectively. As shown in photomicrographs of H&E stained specimens, various degrees of vacuolization at the injury site (black arrow) were observed in most tissues. Severe vacuolization was demonstrated in the control group at day 7, which was improved as recovery progressed. This vacuolization, representing loss of neurons, was greatly reduced in the AgNPs-treated group relative to the control group at each time point. Similarly, at each observation time point there was less myelin loss in AgNPs-treated rats in comparison with rats in the control group.

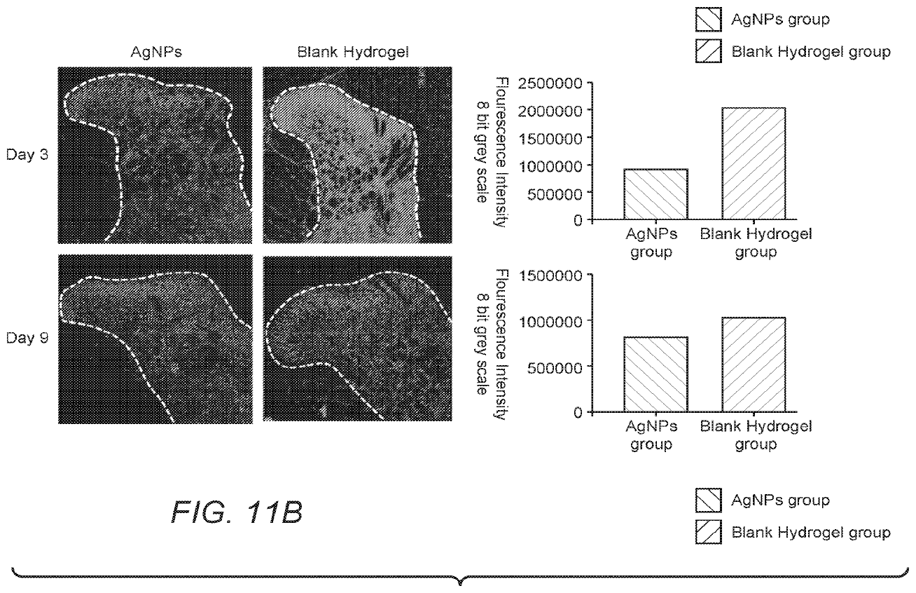

Immunohistochemistry assessment: FIGS. 11A and 11B show the results of immunohistochemical staining directed to TNF-alpha (FIG. 11A) and to iNOS (FIG. 11B), which are both indicators of inflammation. FIG. 11A shows typical results of immunofluorescence staining using a primary antibody directed to TNF-alpha, at the dorsal horn of spinal cord lesion sites in AgNPs group and blank hydrogel group at day 3 and day 9 at a magnification of 10×. FIG. 11B shows typical results of similar immunofluorescent staining studies in which the primary antibody is directed to iNOS. The mean intensity of fluorescence was quantified by software Image J. Values shown are means with standard deviation.

It is apparent that application of silver nanoparticles significantly reduces the expression of inflammatory factors TNF-alpha and iNOS after spinal cord injury, indicating that secondary injury is at least in part attenuated by a reduction of inflammation following such treatment.

Inventors have found, using a contusive injury animal model to demonstrate TSCI, that silver nanoparticles have a significant positive impact on recovery from the injury on both a histological and functional basis. As shown, AgNPs treated subjects show a better and faster recovery as shown using a Forelimb Locomotor Scale (FLS) test at 7th day, 9th day and 14th day post injury and a Ladder rung walking test at 5th day, 7th day, 9th day and 14th day post injury. This functional assessment demonstrates that AgNPs can provide positive influence in the recovery of the spinal cord after injury.

The results of histological examination also indicate that post-injury damage to the traumatized spinal cord is less severe in AgNPs treated subjects than control subjects, as evidenced by reduced vacuolization and reduced demyelination. The results of immunohistochemistry examination showed that the inflammation is substantially reduced as evidenced by the reduction of TNF-alpha and iNOS expression after the injury. This demonstrates that AgNPs can successfully reduce the secondary damage after TSCI. Inventors believe that modulation of M1/M2 ratio by silver nanoparticles released post-injury resulting in improved healing and functional recovery following spinal cord injury.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

### REFERENCES

1. Bracken M B, Shepard M J, Hellenbrand K G, Collins W F, Leo L S, Freeman D F, et al. Methylprednisolone and neurological function 1 year after spinal cord injury. Results of the National Acute Spinal Cord Injury Study. J Neurosurg. 1985; 63(5):704-13.
2. Bracken M B, Shepard M J, Holford T R, Leo-Summers L, Aldrich E F, Fazl M, et al. Administration of methylprednisolone for 24 or 48 hours or tirilazad mesylate for 48 hours in the treatment of acute spinal cord injury. Results of the Third National Acute Spinal Cord Injury Randomized Controlled Trial. National Acute Spinal Cord Injury Study. JAMA. 1997; 277(20):1597-604.
3. Young W, Bracken M B. The Second National Acute Spinal Cord Injury Study. J Neurotrauma. 1992; 9 Suppl 1:S397-405.
4. Nagoshi N, Nakashima H, Fehlings M G. Riluzole as a neuroprotective drug for spinal cord injury: from bench to bedside. Molecules. 2015; 20(5):7775-89.
5. Wilson J R, Fehlings M G. Riluzole for acute traumatic spinal cord injury: a promising neuroprotective treatment strategy. World Neurosurg. 2014; 81(5-6):825-9.
6. Fehlings M G, Kopjar B, Grossman R G. 329 Efficacy and Safety of Riluzole in Acute Spinal Cord Injury: Rationale and Design of AOSpine Phase III Multicenter Double-Blinded Randomized Controlled Trial (RISCIS). Neurosurgery. 2016; 63 Suppl 1:196.
7. Chaloupka K, Malam Y, Seifalian A M. Nanosilver as a new generation of nanoproduct in biomedical applications. Trends in biotechnology. 2010; 28(11):580-8.
8. Ding T, Lu W W, Zheng Y, yang Li Z, bo Pan H, Luo Z. Rapid repair of rat sciatic nerve injury using a nanosilver-embedded collagen scaffold coated with laminin and fibronectin. Regenerative medicine. 2011; 6(4):437-47.
9. Kwan K H, Liu X, To M K, Yeung K W, Ho C-m, Wong K K. Modulation of collagen alignment by silver nanoparticles results in better mechanical properties in wound healing. Nanomedicine: Nanotechnology, Biology and Medicine. 2011; 7(4):497-504.
10. Thirumurugan G, Veni V S, Ramachandran S, Seshagiri Rao J, Dhanaraju M. Superior wound healing effect of topically delivered silver nanoparticle formulation using eco-friendly potato plant pathogenic fungus: synthesis and characterization. Journal of biomedical nanotechnology. 2011; 7(5):659-66.
11. Zhang R, Lee P, Lui V C, Chen Y, Liu X, Lok C N, et al. Silver nanoparticles promote osteogenesis of mesenchymal stem cells and improve bone fracture healing in osteogenesis mechanism mouse model. Nanomedicine. 2015.
12. Kwan K H, Yeung K W, Liu X, Wong K K, Shum H C, Lam Y W, et al. Silver nanoparticles alter proteoglycan expression in the promotion of tendon repair. Nanomedicine. 2014; 10(7):1375-83.
13. Kwan K H, Liu X, To M K, Yeung K W, Ho C M, Wong K K. Modulation of collagen alignment by silver nanoparticles results in better mechanical properties in wound healing. Nanomedicine. 2011; 7(4):497-504.
14. Tian J, Wong K K, Ho C M, Lok C N, Yu W Y, Che C M, et al. Topical delivery of silver nanoparticles promotes wound healing. ChemMedChem. 2007; 2(1):129-36.
15. Wong K K, Cheung S O, Huang L, Niu J, Tao C, Ho C M, et al. Further Evidence of the Anti-inflammatory Effects of Silver Nanoparticles. ChemMedChem. 2009; 4(7):1129-35.
16. Kwan K H, Yeung K W, Liu X, Wong K K, Shum H C, Lam Y W, et al. Silver nanoparticles alter proteoglycan expression in the promotion of tendon repair. Nanomedicine: Nanotechnology, Biology and Medicine. 2013.
17. Stramer B M, Mori R, Martin P. The inflammation-fibrosis link? A Jekyll and Hyde role for blood cells during wound repair. Journal of Investigative Dermatology. 2007; 127(5):1009-17.
18. Liu X, Lee Py, Ho Cm, Lui V C, Chen Y, Che Cm, et al. Silver nanoparticles mediate differential responses in keratinocytes and fibroblasts during skin wound healing. ChemMedChem. 2010; 5(3):468-75.
19. Dziendzikowska K, Gromadzka-Ostrowska J, Lankoff A, Oczkowski M, Krawczyńska A, Chwastowska J, et al. Time-dependent biodistribution and excretion of silver nanoparticles in male Wistar rats. Journal of Applied Toxicology. 2012; 32(11):920-8.
20. Lankveld D, Oomen A, Krystek P, Neigh A, Troost-de Jong A, Noorlander C, et al. The kinetics of the tissue distribution of silver nanoparticles of different sizes. Biomaterials. 2010; 31(32):8350-61.
21. Calandra T, Roger T. Macrophage migration inhibitory factor: a regulator of innate immunity. Nat Rev Immunol. 2003; 3(10):791-800.
22. Tator C H. Update on the pathophysiology and pathology of acute spinal cord injury. Brain Pathology. 1995; 5(4):407-13.
23. Sekhon L H, Fehlings M G. Epidemiology, demographics, and pathophysiology of acute spinal cord injury. Spine. 2001; 26(24S):S2-S12.
24. Kraus J F. Injury to the head and spinal cord. The epidemiological relevance of the medical literature published from 1960 to 1978. Journal of neurosurgery. 1980:S3-10.
25. Carter Jr R. Etiology of traumatic spinal cord injury: statistics of more than 1,100 cases. Texas medicine. 1977; 73(6):61-5.
26. Albin M S, White R. Epidemiology, physiopathology, and experimental therapeutics of acute spinal cord injury. Critical care clinics. 1987; 3(3):441-52.
27. Basso D M, Beattie M S, Bresnahan J C. Graded histological and locomotor outcomes after spinal cord contusion using the NYU weight-drop device versus transection. Experimental neurology. 1996; 139(2):244-56.
28. Carlson S L, Parrish M E, Springer J E, Doty K, Dossett L. Acute inflammatory response in spinal cord following impact injury. Experimental neurology. 1998; 151(1):77-88.
29. Balentine J. Pathology of experimental spinal cord trauma. I. The necrotic lesion as a function of vascular injury. Laboratory investigation; a journal of technical methods and pathology. 1978; 39(3):236-53.
30. KWO S, YOUNG W, DECRESCITO V. Spinal cord sodium, potassium, calcium, and water concentration changes in rats after graded contusion injury. Journal of neurotrauma. 1989; 6(1):13-24.
31. Wrathall J R, Teng Y D, Choiniere D. Amelioration of Functional Deficits from Spinal Cord Trauma with Systemically Administered NBQX, an Antagonist of Non-<i>N</i>-methyl-D-aspartate receptors. Experimental neurology. 1996; 137(1):119-26.
32. Dumont R J, Okonkwo D O, Verma S, Hurlbert R J, Boulos P T, Ellegala D B, et al. Acute spinal cord injury, part I: pathophysiologic mechanisms. Clinical neuropharmacology. 2001; 24(5):254-64.
33. Wolman L. The disturbance of circulation in traumatic paraplegia in acute and late stages: a pathological study. Spinal Cord. 1965; 2(4):213-26.
34. Young W. Secondary injury mechanisms in acute spinal cord injury. The Journal of emergency medicine. 1992; 11:13-22.
35. Anderson D K, Hall E D. Pathophysiology of spinal cord trauma. Annals of emergency medicine. 1993; 22(6):987-92.
36. Geisler F H, Dorsey F C, Coleman W P. Recovery of motor function after spinal-cord injury—a randomized, placebo-controlled trial with GM-1 ganglioside. New England Journal of Medicine. 1991; 324(26):1829-38.
37. Lapchak P A, Araujo D M, Song D, Wei J, Zivin J A. Neuroprotective Effects of the Spin Trap Agent Disodium-[(tert-butylimino) methyl] benzene-1, 3-disulfonate N-Oxide (Generic NXY-059) in a Rabbit Small Clot Embolic Stroke Model Combination Studies With the Thrombolytic Tissue Plasminogen Activator. Stroke. 2002; 33(5):1411-5.
38. Blight A R, Young W. Central axons in injured cat spinal cord recover electrophysiological function following remyelination by Schwann cells. Journal of the neurological sciences. 1989; 91(1):15-34.
39. Fleming J C, Norenberg M D, Ramsay D A, Dekaban G A, Marcillo A E, Saenz A D, et al. The cellular inflammatory response in human spinal cords after injury. Brain. 2006; 129(12):3249-69.
40. Ren Y, Young W. Managing Inflammation after spinal cord injury through manipulation of macrophage function. Neural plasticity. 2013; 2013.
41. Shechter R, Miller 0, Yovel G, Rosenzweig N, London A, Ruckh J, et al. Recruitment of beneficial M2 macrophages to injured spinal cord is orchestrated by remote brain choroid plexus. Immunity. 2013; 38(3):555-69.
42. Mantovani A, Bussolino F, Dejana E. Cytokine regulation of endothelial cell function. The FASEB Journal. 1992; 6(8):2591-9.
43. Shappell S, Smith C. Acute inflammatory response: granulocyte migration and activation. The Handbook of Immunopharmacology: Adhesion Molecules. 1994:29-42.
44. Stoolman L M. Adhesion molecules controlling lymphocyte migration. Cell. 1989; 56(6):907-10.
45. Mackay C R, Imhof B A. Cell adhesion in the immune system. Immunology today. 1993; 14(3):99-102.
46. Bao F, Chen Y, Dekaban G A, Weaver L C. Early anti-inflammatory treatment reduces lipid peroxidation and protein nitration after spinal cord injury in rats. Journal of neurochemistry. 2004; 88(6):1335-44.
47. Soares H D, Hicks R R, Smith D, McIntosh T K Inflammatory leukocytic recruitment and diffuse neuronal degeneration are separate pathological processes resulting from traumatic brain injury. The Journal of neuroscience. 1995; 15(12):8223-33.
48. Kaczorowski S L, Schiding J K, Toth C A, Kochanek P M. Effect of soluble complement receptor-1 on neutrophil accumulation after traumatic brain injury in rats. Journal of Cerebral Blood Flow & Metabolism. 1995; 15(5):860-4.
49. Barone F, Hillegass L, Tzimas M, Schmidt D, Foley J, White R, et al. Time-related changes in myeloperoxidase activity and leukotriene B4 receptor binding reflect leukocyte influx in cerebral focal stroke. Molecular and chemical neuropathology. 1995; 24(1):13-30.
50. Clark R, Lee E, White R, Jonak Z, Feuerstein G, Barone F. Reperfusion following focal stroke hastens inflammation and resolution of ischemic injured tissue. Brain research bulletin. 1994; 35(4):387-92.
51. Perry V, Brown M, Gordon S. The macrophage response to central and peripheral nerve injury. A possible role for macrophages in regeneration. The Journal of experimental medicine. 1987; 165(4):1218-23.
52. David S, Bouchard C, Tsatas 0, Giftochristos N. Macrophages can modify the nonpermissive nature of the adult mammalian central nervous system. Neuron. 1990; 5(4):463-9.
53. Zhang R, Chopp M, Li Y, Zaloga C, Jiang N, Jones M, et al. Anti-ICAM-1 antibody reduces ischemic cell damage after transient middle cerebral artery occlusion in the rat. Neurology. 1994; 44(9):1747-.
54. Jiang N, Moyle M, Soule H R, Rote W E, Chopp M. Neutrophil inhibitory factor is neuroprotective after focal ischemia in rats. Annals of neurology. 1995; 38(6):935-42.
55. Garcia J H, Liu K-F, Relton J K. Interleukin-1 receptor antagonist decreases the number of necrotic neurons in rats with middle cerebral artery occlusion. The American journal of pathology. 1995; 147(5):1477.
56. Connolly Jr E S, Winfree C J, Springer T A, Naka Y, Liao H, Yan S, et al. Cerebral protection in homozygous null ICAM-1 mice after middle cerebral artery occlusion. Role of neutrophil adhesion in the pathogenesis of stroke. Journal of Clinical Investigation. 1996; 97(1):209.
57. CLARK R S, CARLOS T M, SCHIDING J K, BREE M, FIREMAN L A, DeKOSKY S T, et al. Antibodies against Mac-1 attenuate neutrophil accumulation after traumatic brain injury in rats. Journal of neurotrauma. 1996; 13(6):333-41.
58. Chen H, Chopp M, Zhang R, Bodzin G, Chen Q, Rusche J, et al. Anti-CD11b monoclonal antibody reduces ischemic cell damage after transient focal cerebral ischemia in rat. Annals of neurology. 1994; 35(4):458-63.
59. Chopp M, Zhang R L, Chen H, Li Y, Jiang N, Rusche J R. Postischemic administration of an anti-Mac-1 antibody reduces ischemic cell damage after transient middle cerebral artery occlusion in rats. Stroke. 1994; 25(4):869-75.
60. Popovich P G, Guan Z, Wei P, Huitinga I, van Rooijen N, Stokes B T. Depletion of hematogenous macrophages promotes partial hindlimb recovery and neuroanatomical repair after experimental spinal cord injury. Experimental neurology. 1999; 158(2):351-65.
61. Popovich P, Guan Z, McGaughy V, Fisher L, Hickey W, Basso D. The neuropathological and behavioral consequences of intraspinal microglial/macrophage activation. Journal of Neuropathology & Experimental Neurology. 2002; 61(7):623-33.
62. Rabchevsky A G, Streit W J. Role of microglia in postinjury repair and regeneration of the CNS. Mental retardation and developmental disabilities research reviews. 1998; 4(3):187-92.
63. Schwartz M. Macrophages and Microglia in Central Nervous System Injury&colon; Are They Helpful or Harmful? Journal of Cerebral Blood Flow & Metabolism. 2003; 23(4):385-94.
64. Murray P J, Wynn T A. Protective and pathogenic functions of macrophage subsets. Nature Reviews Immunology. 2011; 11(11):723-37.
65. Kigerl K A, Gensel J C, Ankeny D P, Alexander J K, Donnelly D J, Popovich P G. Identification of two distinct macrophage subsets with divergent effects causing either neurotoxicity or regeneration in the injured mouse spinal cord. The Journal of Neuroscience. 2009; 29(43):13435-44.
66. Gordon S, Taylor P R. Monocyte and macrophage heterogeneity. Nature Reviews Immunology. 2005; 5(12):953-64.
67. Bellora F, Castriconi R, Dondero A, Reggiardo G, Moretta L, Mantovani A, et al. The interaction of human natural killer cells with either unpolarized or polarized macrophages results in different functional outcomes. Proceedings of the National Academy of Sciences. 2010; 107(50):21659-64.
68. Dalli J, Serhan C. Specific lipid mediator signatures of human phagocytes: microparticles stimulate macrophage efferocytosis and pro-resolving mediators. Blood. 2012:blood-2012-04-423525.
69. Guerrero A R, Uchida K, Nakajima H, Watanabe S, Nakamura M, Johnson W, et al. Blockade of interleukin-6 signaling inhibits the classic pathway and promotes an alternative pathway of macrophage activation after spinal cord injury in mice. J Neuroinflammation. 2012; 9(1):40.
70. ABE Y, YAMAMOTO T, SUGIYAMA Y, WATANABE T, SAITO N, KAYAMA H, et al. Apoptotic cells associated with Wallerian degeneration after experimental spinal cord injury: a possible mechanism of oligodendroglial death. Journal of neurotrauma. 1999; 16(10):945-52.
71. Li G L, Farooque M, Holtz A, Olsson Y. Apoptosis of oligodendrocytes occurs for long distances away from the primary injury after compression trauma to rat spinal cord. Acta neuropathologica. 1999; 98(5):473-80.
72. Novak M L, Koh T J. Macrophage phenotypes during tissue repair. Journal of leukocyte biology. 2013; 93(6):875-81.
73. Mosser D M, Edwards J P. Exploring the full spectrum of macrophage activation. Nature Reviews Immunology. 2008; 8(12):958-69.
74. Altschul R L, Theise N D, Rapkin M, O'brien R. Combination Therapy. Google Patents; 2012.
75. Nishio Y, Koda M, Hashimoto M, Kamada T, Koshizuka S, Yoshinaga K, et al. Deletion of macrophage migration inhibitory factor attenuates neuronal death and promotes functional recovery after compression-induced spinal cord injury in mice. Acta neuropathologica. 2009; 117(3):321-8.
76. Bloom B R, Shevach E. Requirement for T cells in the production of migration inhibitory factor. The Journal of experimental medicine. 1975; 142(5):1306-11.
77. Bernhagen J, Calandra T, Mitchell R, Martin S, Tracey K, Voelter W, et al. MIF is a pituitary-derived cytokine that potentiates lethal endotoxaemia. 1993.
78. Calandra T, Roger T. Macrophage migration inhibitory factor: a regulator of innate immunity. Nature reviews immunology. 2003; 3(10):791-800.
79. Ogata A, Nishihira J, Suzuki T, Nagashima K, Tashiro K. Identification of macrophage migration inhibitory factor mRNA expression in neural cells of the rat brain by in situ hybridization. Neuroscience letters. 1998; 246(3):173-7.
80. Koda M, Nishio Y, Hashimoto M, Kamada T, Koshizuka S, Yoshinaga K, et al. Up-regulation of macrophage migration-inhibitory factor expression after compression-induced spinal cord injury in rats. Acta neuropathologica. 2004; 108(1):31-6.
81. Fujimoto S. [Identification of macrophage migration inhibitory factor (MIF) in rat spinal cord and its kinetics on experimental spinal cord injury]. [Hokkaido igaku zasshi] The Hokkaido journal of medical science. 1997; 72(4):409-30.
82. Santos L L, Morand E F. The role of macrophage migration inhibitory factor in the inflammatory immune response and rheumatoid arthritis. Wiener Medizinische Wochenschrift. 2006; 156(1-2):11-8.
83. Plesnila N, Von Baumgarten L, Retiounskaia M, Engel D, Ardeshiri A, Zimmermann R, et al. Delayed neuronal death after brain trauma involves p53-dependent inhibition of NF-KB transcriptional activity. Cell Death & Differentiation. 2007; 14(8):1529-41.
84. Calandra T, Bernhagen J, Metz C N, Spiegel L A, Bacher M, Donnelly T, et al. MIF as a glucocorticoid-induced modulator of cytokine production. Nature. 1995; 377(6544):68-71.
85. Gruner J A. A monitored contusion model of spinal cord injury in the rat. J Neurotrauma. 1992; 9(2):123-6; discussion 6-8.
86. Basso D M, Beattie M S, Bresnahan J C. Graded histological and locomotor outcomes after spinal cord contusion using the NYU weight-drop device versus transection. Exp Neurol. 1996; 139(2):244-56.
87. Singh A, Krisa L, Frederick K L, Sandrow-Feinberg H, Balasubramanian S, Stackhouse S K, et al. Forelimb locomotor rating scale for behavioral assessment of recovery after unilateral cervical spinal cord injury in rats. J Neurosci Methods. 2014; 226:124-31.
88. Metz G A, Whishaw I Q. The ladder rung walking task: a scoring system and its practical application. J Vis Exp. 2009(28).

1-87. (canceled)

88. A method of providing a treatment of a neuronal tissue, comprising:
providing a silver nanoparticle preparation comprising silver nanoparticles; and
applying the silver nanoparticle preparation to a site in need of treatment.

89. The method of claim 88, wherein the treatment comprises treatment of a neuronal injury following damage to the neuronal tissue.

90. The method of claim 88, wherein the treatment comprises providing neuroprotection prior to damage to the neuronal tissue.

91. The method of claim 88, wherein the site in need of treatment is selected from the group consisting of a site of an acute spinal cord injury, a site of a head injury, a site of injury to a nerve, and a site of stroke.

92. The method of claim 88, further comprising the step of administering a complementary therapeutic selected from the group consisting of arginase, a corticosteroid, an antibody, and a cytokine.

93. A method of modulating an M1/M2 macrophage balance, comprising:
providing a silver nanoparticle preparation comprising silver nanoparticles; and
applying the silver nanoparticle preparation to a site in need of a treatment providing protection from inflammation, wherein silver nanoparticles are provided in an amount effective to modulate M1/M2 macrophage balance.

94. The method of claim 93, wherein the treatment comprises treatment of a neuronal injury following damage to the neuronal tissue.

95. The method of claim 93, wherein the treatment comprises providing neuroprotection prior to damage to the neuronal tissue.

96. The method of claim 93, wherein the site in need of protection from inflammation is selected from the group consisting of a site of an acute spinal cord injury, a site of a head injury, a site of injury to a nerve, and a site of stroke

97. The method of claim 93, wherein the method comprises application that is localized to at or near the site in need of protection from inflammation.

98. The method of claim 93, further comprising the step of administering a complementary therapeutic selected from the group consisting of arginase, a corticosteroid, an antibody, and a cytokine.

99. A composition for use in treating an animal, comprising:
a plurality of silver nanoparticles; and
a pharmaceutical carrier in the form of a non-flowable gel.

100. The composition of claim 99, wherein the silver nanoparticles have a mean diameter of less than about 1 μm.

101. The composition of claim 99, wherein the silver nanoparticles have a mean diameter of from about 5 nm to about 20 nm.

102. The composition of claim 99, wherein the pharmaceutical carrier comprises a biopolymer.

103. The composition of claim 99, wherein the pharmaceutical carrier further comprises a stabilizing agent, wherein the stabilizing agent is formulated to prevent aggregation of the silver nanoparticles.

104. The composition of claim 99, further comprising a complementary therapeutic selected from the group consisting of arginase, a corticosteroid, an antibody, and a cytokine.

105. A method of treating a site in need of protection from inflammation, comprising:
providing a silver nanoparticle preparation comprising silver nanoparticles; and
applying the silver nanoparticle preparation and arginase to the site in need of a treatment that provides protection from inflammation,
wherein arginase and the silver nanoparticle preparation in combination provide a synergistic effect in protecting the site in need of protection from inflammation.

106. The method of claim 105, wherein the method comprises treatment of a neuronal injury following damage to the neuronal tissue.

107. The method of claim 105, wherein the method comprises providing neuroprotection prior to damage to the neuronal tissue.

108. The method of one of claims 105, wherein the site in need of protection from inflammation is selected from the group consisting of a site of an acute spinal cord injury, a site of a head injury, a site of injury to a nerve, and a site of stroke

109. The method of claim 105, wherein the method comprises application that is localized to at or near the site in need of protection from inflammation.

110. The method of claim 105, further comprising the step of administering a complementary therapeutic selected from the group consisting of a corticosteroid, an antibody, and a cytokine.
